(19) United States
(12) Patent Application Publication
(10) Pub. No.: US 2025/0232873 A1
(43) Pub. Date: Jul. 17, 2025

(54) System and method for screening and diagnosis of adenoma and colorectal cancer

(71) Applicants: Anirban Bhaduri, Bangalore (IN); Ashes Dhanna Ganguly, Bangalore (IN)

(72) Inventors: Anirban Bhaduri, Bangalore (IN); Ashes Dhanna Ganguly, Bangalore (IN)

(73) Assignees: Anirban Bhaduri, Bangalore (IN); Ashes Dhanna Ganguly, Bangalore (IN)

(21) Appl. No.: 18/412,493

(22) Filed: Jan. 13, 2024

(51) Int. Cl. G16H 50/20; G16B 30/10; G16B 35/10; G16H 15/00; G16H 50/70

(52) U.S. Cl. CPC G16H 50/20; G16B 30/10; G16B 35/10; G16H 15/00; G16H 50/70

(57) ABSTRACT

Methods and apparatus for screening and diagnosing a plurality of samples and classifying the same as normal, adenoma and colorectal cancer are disclosed. The methods enable screening and diagnosis of a plurality of samples based on microorganism residing in the gut. The obtained microorganism content and its abundance from a plurality of samples are mapped against a dataset of microorganisms and their abundances stored in a knowledgebase and processed using a preferred methodology to obtain the classification, thereby enabling the screening and diagnosing of, adenoma and colorectal cancer.

### PRIOR ART

Table of prior art

| Publication number | Priority Date | Assignee | Title |
|---|---|---|---|
| U.S. Pat. No. | 2016 Apr. 13 | Psomagen Inc | Method and system for microbiome- |
| 10,265,009B2 | | | derived diagnostics and |
| | | | therapeutics for conditions |
| | | | associated with microbiome |
| | | | taxonomic features |
| EP2955232 | 2014 Jun. 12 | Bork et al., | Method for diagnosing adenomas |
| | | | and/or colorectal cancer |
| | | | (CRC) based on analyzing the |
| | | | gut microbiome |
| US20180258495A1 | 2016 Oct. 6 | Nantes, University of | Method to detect colon cancer |
| | | University of Minnesota | by means of the microbiome |
| CN110637097A | 2018 Mar. 16 | Baylor College of | Identification of combined |
| | | Medicine Second | biomarkers for colorectal |
| | | Genome Inc | cancer using sequence-based |
| | | | excreta microflora survey data |
| U.S. Pat. No. | 2015 Dec. 11 | Mayo Foundation | Compositions and methods for |
| 10,011,878B2 | | for Medical Education | performing methylation |
| | | and Research Exact | detection assays |
| | | Sciences Corp | |

### BACKGROUND OF THE INVENTION

At least one specification heading is required. Please delete this heading section if it is not applicable to your application. For more information regarding the headings of the specification, please see MPEP 608.01(a).

### FIELD

The present disclosure related to methods for screening and diagnosing of adenoma and colorectal cancer and more particularly, to methods of and apparatus for assigning a biological sample into one of the classes of adenoma and colorectal cancer and normal thereof, based on an assessment against a curated knowledgebase, comprising of predefined dataset of microorganisms and their abundances obtained from previously processed samples.

### DESCRIPTION OF THE RELATED ART

There are multiple methods to screen and diagnose adenoma and colorectal cancer. The most common being Fecal Occult Blood Test (FOBT), Fecal Immunological Test (FIT) and endoscopy. The FOBT and the FIT are non-invasive and ideal for screening, however they are not specific. Endoscopy, though sensitive and specific is not effective for screening owing to its invasive nature.

Recent development suggests that genetic panel tests, DNA methylation status test, microbiome composition and glycoproteins are good predictors of adenoma and colorectal cancers. These approaches are more specific as compared to the protein marker-based test of FOBT and fecal immunological tests, but will lack its transferability to complex and diverse population of data. The complexity being a result of the high dimensionality and interdependency of the variables. It should be pointed out that the problem of addressing the high dimensionality and interdependency of the variables is not a solved problem for performing screening and diagnosis based on microbiome or glycoprotein samples obtained from adenoma and colorectal cancer patients.

### SUMMARY OF THE INVENTION

At least one specification heading is required. Please delete this heading section if it is not applicable to your application. For more information regarding the headings of the specification, please see MPEP 608.01(a).

Provided are methods and apparatuses for screening and diagnosing of adenoma and colorectal cancer.

Provided is a non-transitory computer-readable storage medium having recorded thereon a program for causing a computer to execute the methods described herein. The technical problems to be solved by the present embodiments are not limited to the technical problems described above; yet, another technical problem can be inferred from the following embodiments.

Additional aspects will be set forth in part in the description which follows and, in part, will be apparent from the description, or may be learned by practice of the presented exemplary embodiments.

According to an aspect of an exemplary embodiment, a method of screening and diagnosing of adenoma and colorectal cancer, that includes processing a plurality of samples as input data wherein the input data comprises at least one of fecal data,

In one of the exemplary embodiments, the processing of the input data involves identifying a set of microorganisms within the input data, mapping of the input data against a dataset of microorganism and their abundances stored in a knowledgebase and processed using a preferred methodology to obtain the classification. The classification can be at least one of, the normal sample, adenoma sample and the colorectal cancer sample.

The processing of input data may include at least one of the preferred methodology, Logistic Regression (LR) and Random Forest (RF) and Gradient Boosting Model (GBM) and Adaptive Boosting model (ABM)

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF DRAWINGS

These and/or other aspects will become apparent and more readily appreciated from the following description of the exemplary embodiments, taken in conjunction with the accompanying drawings in which:

FIG. 1 illustrates a flowchart to diagnose adenoma, colorectal cancer and normal samples, according to an embodiment.

FIG. 2. is a flowchart illustrating a method of assessing and mapping an input sample against a dataset of microorganism and their abundances stored in a knowledgebase and a preferred methodology to obtain the classification.

FIG. 3. Is a flowchart illustrating a method to select a preferred methodology mapped to the dataset and processing the sample input data to compute the probability of the input data to belong to a class. The class being at least one of, the normal sample, adenoma sample and the colorectal cancer sample.

FIG. 4A illustrates an exemplary implementation of embodiment of the invention and reporting of its performance against other equivalent state of the art methods for a selected set of annotated samples, comprising of normal samples, adenoma samples and colorectal cancer samples.

FIG. 4B illustrates an exemplary implementation of embodiment of the invention and reporting of its performance against other equivalent methods of random set of 100 annotated samples, comprising of normal samples, adenoma samples and colorectal cancer samples.

FIG. 5. is a block level diagram illustrating the assessment pipeline to diagnose adenoma, colorectal cancer and normal samples, according to an embodiment.

### DETAILED DESCRIPTION OF THE INVENTION

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

The advantages and features of the inventive concept and methods of achieving the advantages and features will be described fully with reference to the accompanying drawings, in which exemplary embodiments of the inventive concept are shown. The inventive concept may, however, be embodied in many different forms and should not be construed as being limited to the exemplary embodiments set forth herein; rather these exemplary embodiments are provided so that this disclosure will be thorough and complete, and will fully convey the concept of the inventive concept to one of ordinary skill in the art.

Most of the terms used herein are general terms that have been widely used in the technical art to which the inventive concept pertains. However, some of the terms used herein may be created to reflect the intentions of technicians in this art, precedents, or new technologies. Also, some of the terms used herein may be arbitrarily chosen by the present applicant. In this case, these terms are defined in detail below. Accordingly, the specific terms used herein should be understood based on the unique meanings thereof and the whole context of the inventive concept.

Throughout the specification, when a portion “includes” or “consists of” an element, another element may be further included, rather than excluding the existence of the other element, unless otherwise described.

Hereinafter, the singular forms “a”, “an” and “the” are intended to include the plural forms as well, unless expressly stated otherwise. For example, a “microorganism”, a “preferred method”, and a “input data sample” may each include at least one microorganism, at least one preferred method approach and at least one input data sample.

Hereinafter, exemplary embodiments will be described in detail with reference to the accompanying drawings. However, the constitution in the embodiments and drawings is merely exemplary, and thus this is not intended to limit the inventive concept to particular modes of practice, and it is to be appreciated that all changes, equivalents, and substitutes that do not depart from the spirit and technical scope of the inventive concept are encompassed in the inventive concept.

According to an exemplary embodiment, a method of and apparatus for assessing input data that includes a fecal sample, and/or a microbiome and/or filtering the input data is provided. The assessment may be performed based on information or data from a knowledgebase.

The knowledgebase may include data sets regarding features such as microorganism composition along with their abundances from human subjects, and/or list of microorganisms and a preferred methodology to obtain the classification of the input data sample associated with the input data sample. In brief, a set of features representing microorganism compositions and their abundances that may be extracted from the knowledgebase and a preferred methodology for processing and classifying the input data. The class being at least one of, the normal sample, adenoma sample and the colorectal cancer sample.

In one aspect, a machine learning application is the preferred method to obtain the classification results. The most commonly used preferred method applicable to the method described herein include, but are not limited to Random Forest (RF), Adaptive Boosting Method (ABM), Gradient Boosting Method (GBM), and Logistic regression (LR). Most preferably, Random Forest (RF), Adaptive Boosting Method (ABM) and Gradient Boosting Method (GBM) is used.

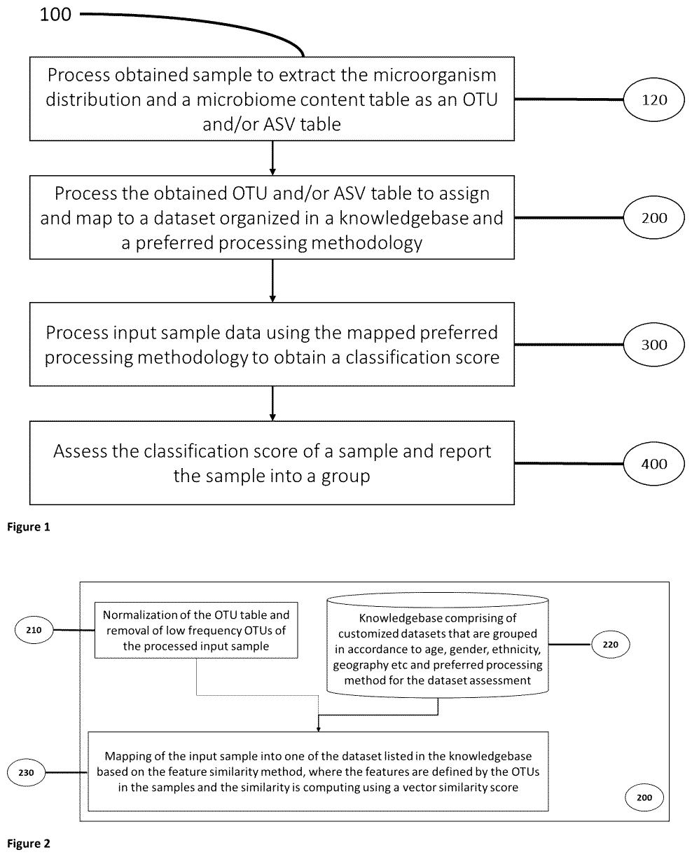

FIG. 1 is a flowchart and illustrates process 100, of a method of assessing input data based on data from a biological sample and assigning a class based on a classification score, according to an embodiment. In operation 120, the input data may be received and processed to identify attributes and features, such as but not limited to, a 16S DNA based microorganism distribution and a microbiome content table, an Operational Taxonomic Units (OTU) table and an Amplicon Sequence Variant (ASV). In operation 200, extracted attributes and features from the input data may be assigned and mapped to a dataset organized in a knowledgebase and a preferred processing methodology through a process of similarity scoring. In operation 300, extracted attributes and features from the input data may be processed against to a mapped dataset from the knowledgebase using a preferred processing methodology to classification score the input data to be classified into one of the selected classes The classes being one of the mentioned but not limited to classes, such as to normal, and adenoma, and colorectal cancer. Finally in operation 400, the input data may be assessed based on the analysis performed and be reported into on the class based on the classification score. The assessment may involve assigning of the input sample into on the mentioned but not limited to classes, such as to normal, and adenoma, and colorectal cancer. Hereinafter, each operation of FIG. 1 will be described in more detail.
1. Processing Input Data (Operation 120)
   The obtained biological samples received as Input Data are to be processed to identify and tabulated as the microbiological composition and the abundance of the microbiological composition within the sample. In an embodiment, the processing may be performed using an 16S sequencing approach. In another embodiment the processing may be performed using a florescence-based detection of the microorganisms and their abundance. Following the detection of the microorganism within the sample the microorganisms are tabulated along with their abundances.
2. Mapping to the input data against a dataset and preferred processing method listed in the knowledgebase (Operation 200).
   The tabulated Input Data of the microorganisms is screened against a knowledgebase and mapped against a dataset and a preferred processing methodology. Operations for mapping the input data set against a dataset and selection of the preferred processing method will be described in detail below with reference to FIG. 2.
3. Processing of the Input Data against the mapped dataset using the preferred processing method listed in the knowledgebase (Operation 300).
   As used herein, the Input Data is processed using the preferred processing method against the selected data set from the knowledgebase and a probability score of the Input Data to be classified into a group is obtained. Operations for scoring the input data set against a dataset and scoring the sample to obtain a classification score will be described in detail below with reference to FIG. 3.
4. Reporting the class of the Input Data based on the classification scores (Operation 400)
   In association with the classification score, in one of the embodiments the Input Data is classified into one of but not limited to the groups, of normal, adenoma and colorectal cancer.

FIG. 2 illustrates the process 200, of mapping of the Input Data to a dataset listed in the knowledgebase, according to an embodiment

In operation 210, extracted attributes and features from the input data may be received as a table recording the microorganism composition and the abundance and processed for normalization of the data. The normalization of the data is performed by at least one the approaches Linear normalization, Z-Score normalization, and Standard Deviation Normalization, Microorganism reporting a normalized abundance greater than a threshold value is considered for further analysis.

In operation 220, receive as a knowledgebase may include customized dataset comprising of microorganism content of a set of samples and a set of preferred processing method for assessment. The customized dataset of microorganism content of a set of samples are grouped in accordance to at least one of the following parameters, age, geography, ethnicity, gender, Sedentary habits, Smoking habits and Dietary habits.

In operation 230, the extracted attributes and features from the input data as obtained from 210 is screened against datasets as received from the operation of 220 and scored for similarity. The scoring of the similarity is computed by representing the microorganism composition and abundance as a linear vector and using at least one of the approaches of Jaccard score, Cosine similarity metric, Hamming distance, Levenshtein distance and Sorensen-Dice for computing and scoring the similarity. Once the similarity scoring computed against plurality of the entries across the plurality of dataset, the dataset reporting the highest similarity is mapped to the input data from which attributes and features are extracted.

FIG. 3 illustrates the process flowchart, 300, for scoring of the of the Input Data as obtained from operation 210 in FIG. 2 against a selected data set listed in the knowledgebase, using a preferred processing methodology to obtain a classification score, according to an embodiment.

In operation 310, receive as a dataset obtained from a knowledgebase and an associated preferred processing methodology.

In operation 320, the input data as obtained from operation 210 in FIG. 2 is score for classification into a group using the associated preferred processing methodology. The scoring is performed using a preferred methodology applicable to the method described herein include, but are not limited to Random Forest (RF), Adaptive Boosting Method (ABM), Gradient Boosting Method (GBM), and Logistic regression (LR). The classified group herein include, but are not limited to are normal, adenoma and colorectal cancer. The scoring is performed using a preferred methodology herein includes a probability of the input data as obtained from operation 210 to be classified into the groups.

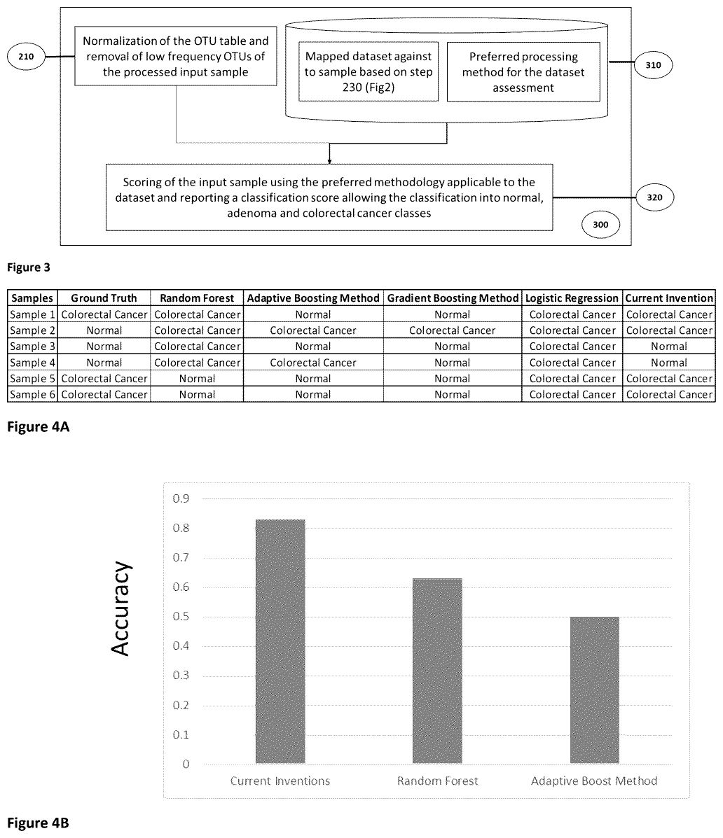

FIGS. 4A and 4B illustrated the example performance results of an implemented embodiment of the method.

FIG. 4A reports the implementation of an embodiment of the current invention and its comparison against equivalent methods such as Random Forest, Adaptive Boosting method, Gradient Boosting method, and logistic regression. The Figure reports the class of the samples using the various methods. As reported in FIG. 4A the current invention outperforms the current approaches such as Random Forest, Adaptive Boosting method, Gradient Boosting method, and logistic regression.

FIG. 4B reports the implementation of an embodiment of the current invention and its comparison a curated dataset of 100 clinical samples and independent performance of the implemented embodiment of the current invention against an equivalent implementation of Random Forest and Adaptive Boosting method. The data set comprised of Normal, Adenoma and Colorectal cancer samples. As reported in FIG. 4B the current method outperforms the current state of the art methods.

As observed the results and recommendations will enable medical practitioners to classify patients and perform clinical diagnosis.

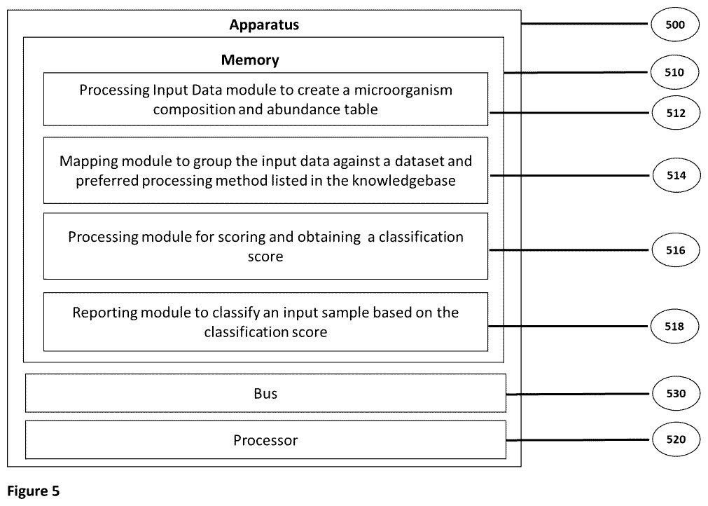

FIG. 5 is a block diagram of an apparatus 500 for assessing the input data based on the knowledgebase and computing the classification score according to an embodiment.

The apparatus 500 may include a processor 520 and a memory 510 coupled to the processor 520 through a bus 530. The processor 520 may include a microprocessor, a microcontroller, a computational circuit, a complex instruction set computing (CISC) microprocessor, a reduced instruction set computing (RISC) microprocessor, a very long instruction word (VLIW) microprocessor, an explicitly parallel instruction computing (EPIC) microprocessor, a digital signal processor, any other type of processing circuit, or a combination thereof.

The memory 510 may include a computer memory element storing at least one module in the form of executable program which, when executed by the processor 520, instructs the processor 520 to perform the method operations illustrated in FIG. 1 to FIG. 3.

The memory 510 may include a Processing Input Data module to create a microorganism composition and abundance table, 512, a mapping module to group the input data against a dataset and preferred processing method listed in the knowledgebase 514, a processing module for scoring and obtaining a classification score 516 and a Reporting module to classify an input sample based on the classification score 518.

Computer memory elements may include any suitable memory devices or storage media for storing data and executable program, such as read only memory (ROM), random access memory (RAM), erasable programmable read only memory (EPROM), electrically erasable programmable read only memory (EEPROM), hard drive, or memory cards.

The apparatus 500 may operate in conjunction with program modules, including functions, procedures, data structures, and application programs, for performing tasks, defining abstract data types, or low-level hardware contexts. Executable program stored on any of the above-mentioned storage media may be executable by the processor 520.

The processing module 512 instructs the processor 520 to necessarily perform operation 120 of FIG. 1.

The processing module 514 instructs the processor 520 to necessarily perform operation 230 of FIG. 2.

The processing module 516 instructs the processor 520 to necessarily perform operation 320 of FIG. 3.

The processing module 518 instructs the processor 520 to necessarily perform operation 400 of FIG. 1.

In FIG. 5, the apparatus 500 is illustrated to have the module 512, module 514, module 516 and module 518 separately, but the while analyzing one or more modules may be merged as a single assessment unit and instruct the processor 520 to perform the necessary operations of 120 of FIG. 1, 230 of FIG. 2, 320 of FIG. 3 and 400 of FIG. 1.

The present embodiments have been described with reference to specific example embodiments; it will be evident that various modifications and changes may be made to these embodiments without departing from the broader spirit and scope of the various embodiments. In other words, claims may be construed as including such replacements, modifications, and changes. Therefore, the content throughout the specification and drawings should be construed in a non-limiting sense.

The device described herein may include a processor, a memory for storing program data and executing it, a permanent storage unit such as a disk drive, a communications port for handling bi-directional communications with external devices (e.g., an internal/directly connected knowledgebase and/or an external/remote knowledgebase), and user interface devices, including a touch panel, keys, buttons, etc. When software modules or algorithms are involved, these software modules may be stored as program instructions or computer readable code executable on a processor on a computer-readable medium. Examples of the computer-readable medium include storage media such as magnetic storage media (e.g., read only memories (ROMs), random-access memory (RAMs), floppy discs, or hard discs), optically readable media (e.g., compact disk-read only memories (CD-ROMs) or digital versatile disks (DVDs)), etc. The computer readable recording medium can also be distributed over network coupled computer systems so that the computer readable code is stored and executed in a distributive manner. This media can be read by the computer, stored in the memory, and executed by the processor.

The exemplary embodiments may be described in terms of functional block components and various processing steps. Such functional blocks may be realized by any number of hardware and/or software components configured to perform the specified functions. For example, the exemplary embodiment may employ various integrated circuit (IC) components, e.g., memory elements, processing elements, logic elements, look-up tables, and the like, which may carry out a variety of functions under the control of one or more microprocessors or other control devices. Similarly, where the elements of the exemplary embodiment are implemented using software programming or software elements, the embodiment may be implemented with any programming or scripting language such as C, C++, Java, assembler language, or the like, with the various algorithms being implemented with any combination of data structures, objects, processes, routines or other programming elements. Functional aspects may be implemented in algorithms that are executed on one or more processors. Furthermore, the present invention could employ any number of conventional techniques for electronics configuration, signal processing and/or control, data processing and the like. The words “mechanism”, “element”, “means”, and “configuration” are used broadly and are not limited to mechanical or physical embodiments, but can include software routines in conjunction with processors, etc. But can include software routines in conjunction with processors, etc.

The particular implementations shown and described herein are illustrative examples of the inventive concept and are not intended to otherwise limit the scope of the inventive concept in any way. For the sake of brevity, conventional electronics, control systems, software development and other functional aspects of the systems may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections may be present in a practical device.

The use of the terms “a” and “an” and “the” and similar referents in the context of describing the inventive concept (especially in the context of the following claims) are to be construed to cover both the singular and the plural. Furthermore, recitation of ranges of values herein are merely intended to serve as a shorthand method of referring individually to each separate value falling within the range, unless otherwise indicated herein, and each separate value is incorporated into the specification as if it were individually recited herein. Also, the steps of all methods described herein can be performed in any suitable order unless otherwise indicated herein or otherwise clearly contradicted by context. The inventive concept is not limited to the described order of the steps. The use of any and all examples, or exemplary language (e.g., “such as”) provided herein, is intended merely to better illuminate the inventive concept and does not pose a limitation on the scope of the inventive concept unless otherwise claimed. Numerous modifications and adaptations will be readily apparent to one of ordinary skill in the art without departing from the spirit and scope.

In addition, other exemplary embodiments can also be implemented through computer readable code and/or instructions stored in or on a medium, e.g., a computer readable medium, to control at least one processing element to implement any above-described exemplary embodiment. The medium can correspond to any medium or media permitting the storage and/or transmission of the computer readable code.

The computer readable code can be recorded/transferred on a medium in a variety of ways, with examples of the medium including recording media, such as magnetic storage media (e.g., ROM, floppy disks, hard disks, etc.) and optical recording media (e.g., CD-ROMs, or DVDs), and transmission media such as Internet transmission media. Thus, the medium may be such a defined and measurable structure including or carrying a signal or information, such as a device carrying a bitstream according to one or more exemplary embodiments. The media may also be a distributed network, so that the computer readable code is stored/transferred and executed in a distributed fashion. Furthermore, the processing element could include a processor or a computer processor, and processing elements may be distributed and/or included in a single device.

It should be understood that the exemplary embodiments described therein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope as defined by the following claims.

1. A method of assessing, screening and diagnosing for at least one of conditions, adenoma and colorectal cancer and normal in one or more samples from one or more subjects, the method comprising:
a sample handling step where the one or more samples from the one or more subjects are processed for at least of the steps, extraction of the nucleic acid content step, multiplexed amplification using one or more primers step, sequencing analysis and alignment step, and recording of the microbiome content report in the sample step;
a classification step where the microorganism content and their abundances report from the one or more samples from the one or more subjects are processed for mapping into the one or more datasets in a knowledgebase using a one or more feature similarity assessment method;
an assessment and scoring step where the microorganism content and their abundances report from the one or more samples from the one or more subjects is processed against the mapped one or more datasets in a knowledgebase using an optimized preferred method approach and reporting as an output a probability of the one or more samples from the one or more subjects to be assigned to at least one of conditions, adenoma and colorectal cancer and normal;
a reporting step, where a report is generated for the one or more samples from the one or more subjects by processing the output of the results of the optimized preferred method approach and recording the one or more samples from the one or more subjects as at least one of conditions, adenoma and colorectal cancer and normal.

2. The method as claimed in claim 1, wherein the classification of the microbiome content report from the one or more samples from the one or more subjects are processed for mapping into the one or more datasets using a feature similarity assessment method based on similarity scoring such as but not limited to Jaccard score, Cosine similarity metric, Hamming distance, Levenshtein distance and Sorensen-Dice metric.

3. The method as claimed in claim 2, wherein the classification of the microorganism content and their abundances report from the one or more samples from the one or more subjects are processed for mapping into the one or more datasets using a feature similarity assessment method, where the features are derived from the prevalence of at least one of the operational taxonomy units and the amplicon sequence variant in the microorganism content and their abundances report.

4. The method as claimed in claim 1, wherein the mapped one or more samples from the one or more subjects are processed for the assessment and scoring using an optimized preferred method learning approach.

5. The method as claimed in claim 4, wherein the mapped machine learning model comprising of but not limited to Logistic Regression (LR) and Random Forest (RF) and Gradient Boosting Model (GBM) and Adaptive Boosting model (ABM).

6. The method as claimed in claim 1, wherein a knowledgebase comprises at least one of the following:
a. customized data sets represented by microbiome content
b. mapped preferred method to the customized data set.

7. The method as claimed in claim 6, wherein the datasets listed within the knowledgebase comprises of customized data sets that are grouped in accordance to at least one of the following parameters:
a. Age
b. Geography
c. Ethnicity
d. Gender
e. Sedentary habit
f. Smoking habit
g. Dietary habit
h. Sequencing Platform.

8. A non-transitory computer readable recording medium having embodied thereon a program for executing a method assessing, screening and diagnosing for at least one of conditions, adenoma and colorectal cancer and normal in one or more samples from one or more subjects wherein execution of the program by at least one processor, causes the at least one processor to:
perform a classification step wherein the microbiome content report from the one or more samples from the one or more subjects are processed to be mapped into the one or more datasets in a knowledgebase using a one or more feature similarity assessment method
perform an assessment and scoring step where the microbiome content report from the one or more samples from the one or more subjects is processed against the mapped one or more datasets in a knowledgebase using an optimized machine learning approach and reporting as an output a probability of the one or more samples from the one or more subjects and assigned to at least one of conditions, adenoma and colorectal cancer and normal
perform a reporting step, where a report is generated for the one or more samples from the one or more subjects by processing the output of the results of the optimized machine learning approach and recording the one or more samples from the one or more subjects as at least one of conditions, adenoma and colorectal cancer and normal.

9. A system for of assessing, screening and diagnosing for at least one of conditions, adenoma and colorectal cancer and normal in one or more samples from one or more subjects, the system the comprising steps of:
a sample handling step where the one or more samples from the one or more subjects are processed for at least of the steps, extraction of the nucleic acid content step, multiplexed amplification using one or more primers step, sequencing analysis and alignment step, and recording of the microbiome content report in the sample step;
a classification step where the microorganism content and their abundances report from the one or more samples from the one or more subjects are processed for mapping into the one or more datasets in a knowledgebase using a one or more feature similarity assessment method;
an assessment and scoring step where the microorganism content and their abundances report from the one or more samples from the one or more subjects is processed against the mapped one or more datasets in a knowledgebase using an optimized preferred method approach and reporting as an output a probability of the one or more samples from the one or more subjects to be assigned to at least one of conditions, adenoma and colorectal cancer and normal;
a reporting step, where a report is generated for the one or more samples from the one or more subjects by processing the output of the results of the optimized preferred method approach and recording the one or more samples from the one or more subjects as at least one of conditions, adenoma and colorectal cancer and normal.

10. The method as claimed in claim 1 wherein subjects are referred to as humans undergoing investigation for at least one conditions, adenoma and colorectal cancer and normal and the normal subjects are humans free from the conditions of adenoma and colorectal cancer.

11. The method as claimed in claim 8 wherein subjects are referred to as humans undergoing investigation for at least one conditions, adenoma and colorectal cancer and normal and the normal subjects are humans free from the conditions of adenoma and colorectal cancer.
